(19) United States
(12) Patent Application Publication
(10) Pub. No.: US 2011/0301549 A1
(43) Pub. Date: Dec. 8, 2011

(54) DEVICE FOR INJECTING LIQUIDS

(75) Inventor: Michael Hartmann, Melsungen (DE)

(73) Assignee: B.BRAUN MELSUNGEN AG, Melsungen (DE)

(21) Appl. No.: 12/522,450

(22) PCT Filed: Jan. 7, 2008
(86) PCT No.: PCT/DE08/00010

§ 371 Date: Nov. 10, 2010

(30) Foreign Application Priority Data
Jan. 9, 2007 (DE) 10 2007 002 096.3

(51) Int. Cl. A61M 5/31; A61M 5/34

(52) U.S. Cl. 604/207; 604/240; 604/241

(57) ABSTRACT

The invention relates to a device for injecting liquids, more particularly liquid medicaments or hormone preparations, for example insulin, said device comprising a housing part in or on which a cartridge containing the liquid can be arranged, wherein the housing part or the cartridge has a needle seat designed for releasable attachment of a needle holder carrying a needle, and wherein the cartridge, upon release of the needle holder from the needle seat, is held fixed in position in or on the housing part. In order to release the needle holder, a release element is provided that engages on the needle holder and that can be activated by an actuating element.

This is a U.S. National Phase application of application number PCT/DE2008/000010, filed Jan. 7, 2008 (which is incorporated herein by reference in its entirety), which claims priority benefit of DE 10 2007 002 096.3 filed Jan. 9, 2007.

### FIELD OF THE INVENTION

The invention relates to a device for injecting liquids, more particularly liquid medicaments or hormone preparations, for example insulin, in accordance with the preamble of claim 1.

### BACKGROUND OF THE INVENTION

A device of this kind is described in EP 0 554 995 B1, for example. In the device designed as an injection pen in said document, the needle seat is provided with an outer thread onto which the needle holder can be screwed, which needle holder has an inner thread and carries a needle. Devices of this kind are often used by persons self-administering certain medicaments, for example insulin. The needles, which should be used only once, for hygiene reasons and because of the associated risk of infection, have to be disposed of and stored away safely after the injection of the medicament.

To allow the needle holder carrying the needle to be released from the needle seat, the needle holder has to be unscrewed from the needle seat. In doing so, the needle holder has to be gripped directly by the fingers, and there is a risk of the exposed needle tip injuring the person unscrewing the needle holder.

Although the needle tip can be provided with a cap, as is shown in EP 0 688 571 B1 for example, the risk of injury still exists, since the needle tip lies exposed when the cap is being fitted.

Particularly in the case of persons self-injecting medicaments in their own homes or in public places, other people are exposed to the inherent risks of used needles, especially if these people themselves are injecting the patient with the medicament or are helping the patient to inject the medicament or to change the needle. In order to minimize the risk of injury and therefore also the risk of infection when releasing the needle holder from the needle seat, disposal containers have been proposed in which a special tool for releasing the needle holder is integrated and in which the used needles can be safely stored. The safe disposal of needles is demanded by the persons using the device and also, in most countries, by legislation.

A disposal container of this kind with an integrated release tool for the needle holder is described, for example, in EP 1 396 234 B1. In order to release the needle holder from the needle seat, the disposal container is provided with an opening that has a special geometry adapted to the needle holder. To release it from the needle seat, the needle holder is placed into this opening and the injection pen is turned, whereupon the needle holder is released from the needle seat by virtue of the corresponding threads. Although the solution proposed in said document is intended to avoid the needle holder becoming jammed in the opening, the possibility of jamming cannot be completely ruled out if the matching geometries of the needle holder and of the opening have error tolerances. The needle holder thus lying firmly in the opening must be pushed through the opening in a second operating step. This can be done using another tool. However, it can also be done by the user, by means of the latter using his or her fingers to try to push the needle holder through the opening into the disposal container. In doing so, the user is of course once again exposed to the inherent risk of injury and infection, since an end of the needle protrudes from the needle holder and thus lies exposed. Moreover, this end of the needle is not covered by a cap or the like since, prior to the release of the needle holder, it was positioned in the cartridge to permit injection of the medicament.

The openings described in EP 1 396 234 B1 are also unable to release needle holders from needle seats if the geometry of the opening is not adapted to the geometry of the needle holder.

### SUMMARY OF THE INVENTION

The problem addressed by the invention is therefore that of making available a device which is of the kind set forth in the preamble of claim 1 and which allows the needle holder to be released from the needle seat easily and without risk, and without the need for a separate tool that is adapted to the geometry of the needle holder and is optionally arranged on a disposal container.

### Invention and Advantages Thereof

This problem is solved by a device having the features of claim 1. Advantageous embodiments of the invention are set forth in the dependent claims.

Since, according to aspects of the invention, the needle holder is released by means of a release element that engages on the needle holder and that can be activated by an actuating element, the person using the device does not require a separate tool to release the needle holder. For the disposal of a used needle, only the device according to the invention and a disposal container are now needed. The geometry of the needle holder does not have to be adapted to the geometry of a separate tool, optionally arranged on the disposal container. Therefore, the person using the device according to the invention is not dependent on specific disposal containers or tools adapted to the device, because the mechanism for releasing the needle holder is a component part of the device itself. Of course, this also greatly enhances the quality of life of the persons who use such devices for self-injection of medicaments, since separate release tools and special disposal containers are no longer required and therefore do not have to be carried around.

According to one embodiment of the invention, the device is designed for manual release of the needle holder. This measure ensures an inexpensive and simple device by means of which the needle holder can be released from the needle seat with the aid of a mechanism that can be activated by hand.

Alternatively, it is of course possible for the device to be designed such that the needle holder is released by motor, more particularly by electric motor. This embodiment of the invention is particularly convenient for the user, because only the actuating element has to be operated in order to activate the release of the needle holder from the needle seat. Manual operation of a mechanism by the user is not necessary, since this is done by motor or electric motor.

It has proven particularly user-friendly if the housing part is substantially pen-shaped or axially symmetrical with respect to the longitudinal axis thereof. This permits comfortable and convenient operation of the device, which is adapted ergonomically to the user's hand.

According to a further concept of the invention, the device as an injection pen has proven particularly useful since such an injection pen can be operated comfortably and conveniently using one hand.

According to a further concept of the invention, the release element is arranged so as to be axially movable on the housing part. This measure has the effect that, upon release of the needle holder, no further space is required in the radial direction of the device according to the invention. The device can thus already be positioned on a disposal container when the needle holder is released from the needle seat, such that the needle holder, after its release, can only escape into the disposal container.

The release element and the actuating element are advantageously connected to each other rigidly, in particular cohesively, as a result of which a complicated mechanism between actuating element and release element can be omitted.

Alternatively, it is also possible, particularly in the case of a release element driven by a motor or electric motor, that the release element and the actuating element are not connected to each other rigidly, but via gear mechanisms. However, such an embodiment requires a more complicated mechanism for it to be possible to release the needle holder from the needle seat.

The release element and/or the actuating element can preferably be moved along a guide on the housing part. The guide can be designed as at least one linear guide rail in the axial direction of the housing part, and elements for engagement in the at least one guide rail are arranged on the release element and/or the actuating element. In this way, the needle holder is released from the needle seat by a simple movement of the release element and/or the actuating element.

However, the guide can also be formed by an outer thread that is arranged on the housing part and that engages with an inner thread of the release element and/or of the actuating element.

In both cases, however, a sleeve that can be moved axially on the housing part can be provided as release element and/or actuating element. A sleeve of this kind does not substantially affect the dimensions of the device, with the result that convenient and comfortable handling of the device according to the invention is still possible.

According to another advantageous embodiment of the invention, the release element for releasing the needle holder engages the latter with a form fit and/or frictional fit and transmits its axial movement to the needle holder, as a result of which the latter is likewise moved in the axial direction of the housing part until it is released from the needle seat. No further parts are needed to release the needle holder. The device is therefore also optimized in terms of the parts used.

The release element can have an annular limit which, in the position of use, engages behind the needle holder arranged on the needle seat. Alternatively, it is of course also possible for the release element to have projections which, upon release of the needle holder from the needle seat, engage in corresponding recesses in the needle holder. Safe and simple release of the needle holder is guaranteed in both cases.

To avoid unintentional release of the needle holder from the cartridge holder, the release element and/or the actuating element can be fixed by at least one fixing element.

Advantageously, according to a further embodiment of the invention, the release element can be transferred back to its starting position by a new, unused needle holder being fitted. However, in a particularly convenient embodiment, the release element is provided with a restoring mechanism, for example a spring force, as a result of which the release element is transferred automatically to its starting position when the needle holder is released from the needle seat.

According to a further embodiment of the invention, the housing part is designed as cartridge holder. The housing part can in this case also have a dosing mechanism. According to a further embodiment of the invention, the cartridge holder and the dosing mechanism can be connected to each other in a releasable manner by means of a coupling.

To allow the filling level of a cartridge made of transparent material to be monitored, it has proven advantageous for the housing part and/or the cartridge holder and/or the sleeve to be made at least partially transparent or to be provided with an opening, for example in the form of a viewing window.

It is also of advantage for the housing part or the cartridge to have coupling means, which correspond with mating coupling means on the needle holder. This measure ensures that the housing part or cartridge can be connected securely to the needle holder. This effectively avoids release of the needle holder from the device according to the invention during the administration or injection of the medicament.

It has proven particularly advantageous if the coupling means are designed as spring elements, and the mating coupling means are designed as recesses which correspond thereto and into which the spring elements engage. Of course, the spring elements can also be formed on static friction surfaces on which the spring elements come to lie, the static friction ensuring a secure fit of the needle holder on the needle seat during the injection.

Alternatively, the coupling means and the mating coupling means can be designed as locking elements and mating locking elements, respectively, or as clip elements and mating clip elements, respectively.

According to a further embodiment of the invention, the coupling means and the mating coupling means are designed as outer and inner threads. In all cases, these embodiments ensure a secure fit of the needle holder on the needle seat during the administration or the injection of the medicament, after which the needle holder can be released from the needle seat in a simple way and without any risk.

In order to protect the entire device from contamination, for hygiene reasons, a protective cap is further provided for the housing part or the cartridge holder.

### BRIEF DESCRIPTION OF DRAWINGS

Further aims, advantages, features and possible uses of the present invention will become clear from the following description of an illustrative embodiment and by reference to the drawings. All of the features described here and/or shown in the drawings represent, either singly or in any desired advantageous combination, the subject matter of the present invention, and do so independently of their presentation in the claims and of the back references indicated in the claims.

FIG. 1 shows a perspective view of an illustrative embodiment of a device according to the invention designed as an injection pen with a protective cap,

FIG. 1a shows the injection pen from FIG. 1 without the protective cap, and with an attached needle holder on which a protective cap is arranged,

FIG. 2 shows the injection pen from Figure la without the protective cap for the needle holder,

FIG. 3 shows an exploded view of the injection pen from FIG. 1,

FIG. 4 shows the injection pen from FIG. 2 in partial section,

FIG. 4a shows a cross-sectional view of a detail from FIG. 4,

FIG. 5 shows a perspective view, in partial section, of the injection pen at a stage during the release of the needle holder from the needle seat,

FIG. 5a shows a cross-sectional view of a detail from FIG. 5,

FIG. 6 shows a perspective view, in partial section, of the injection pen at a stage after the release of the needle holder from the needle seat, and

FIG. 6a shows a cross-sectional view of a detail from FIG. 6.

### DETAILED DESCRIPTION OF THE INVENTION

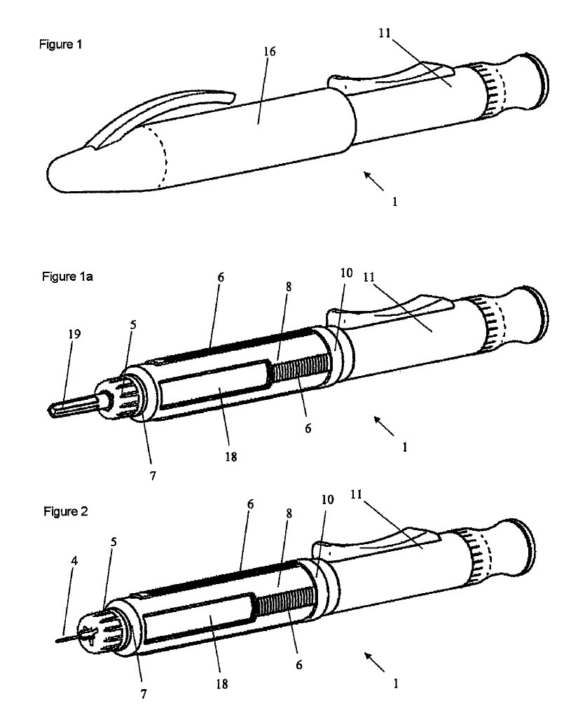

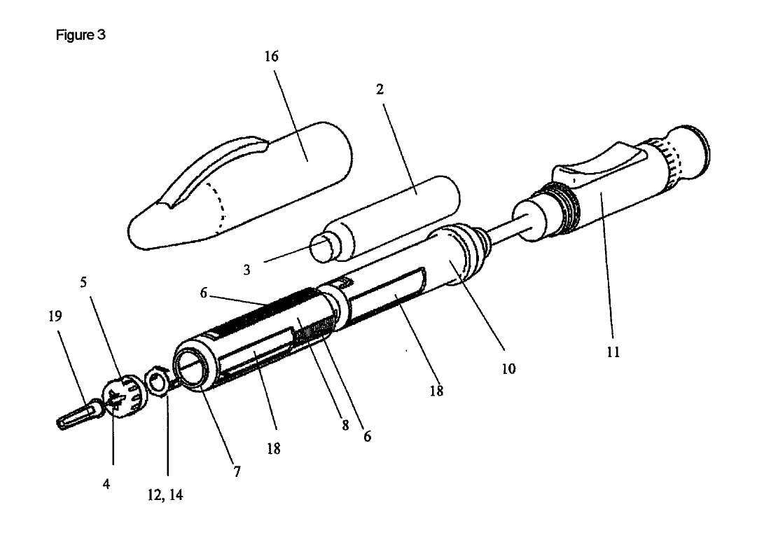

In FIG. 1, an illustrative embodiment of a device according to the invention is shown designed as an injection pen. The injection pen is basically formed by a pen-shaped housing 1, which in the present case comprises a cartridge holder 10 and, connected to the latter in a releasable manner by a thread, a dosing mechanism 11, as is seen in particular from FIG. 2. In FIG. 1, the cartridge holder 10 is covered by a protective cap 16. The cartridge holder 10 is designed to receive a cartridge 2 containing a liquid medicament and having a needle seat 3 at its pierceable, sealed proximal end. A needle holder 5 can be arranged in a releasable manner on the needle seat 3. The needle holder 5 carries a needle 4, which reaches through the pierceable, sealed proximal end of the cartridge 2 into the interior of the latter when the needle holder 5 is arranged on the needle seat 3, as is also shown in FIGS. 1a and 2. In FIG. 1a, the needle 2 of the needle holder 5 is likewise covered by its own protective cap 19.

Coupling means 12 in the form of spring elements 14 are arranged on the needle seat 3 and engage with mating coupling means 13 designed as recesses 15 in the needle holder 5. The spring elements 14 have projections 17, which engage in the recesses 15 of the needle holder 5 when the latter is arranged on the needle seat 3 and thus ensure a secure fit of the needle holder 5 on the needle seat 3 during injection of the medicament.

A sleeve 8 is arranged in a longitudinally movable manner on the cartridge holder 10. The sleeve 8 basically comprises an actuating element 6 and a release element 7, which in the present case are designed in one piece. For moving the sleeve 8 on the cartridge holder 10, a guide for the sleeve 8 is provided. In the described illustrative embodiment, the release element 7 has an annular shape and engages behind the needle holder 5 when the latter is fitted on the needle seat 3.

To allow the filling level of the liquid medicament in the cartridge 2 to be monitored continuously, the cartridge 2 is made of transparent material, and the cartridge holder 10 and sleeve 8 both have viewing windows 18. These at all times ensure a clear view of the contents of the cartridge. For this purpose, it would of course also be possible for both the cartridge holder 10 and sleeve 8 to be made at least partially transparent.

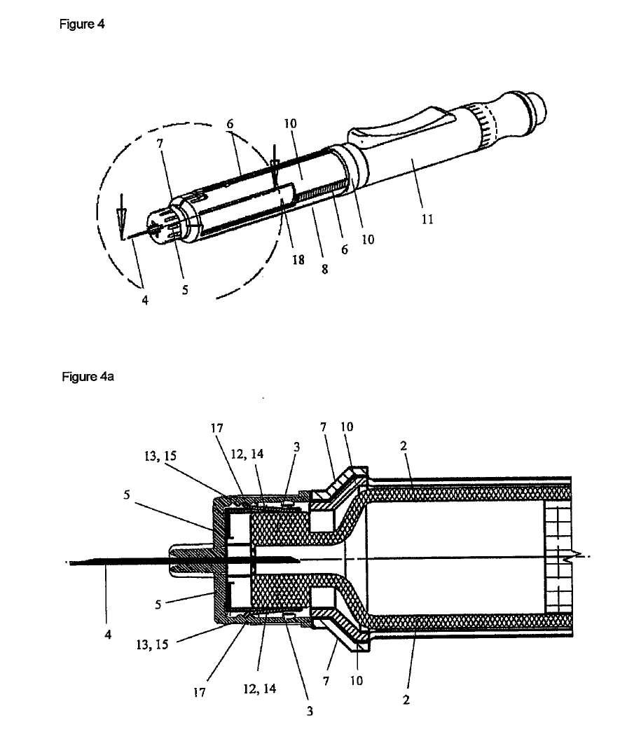

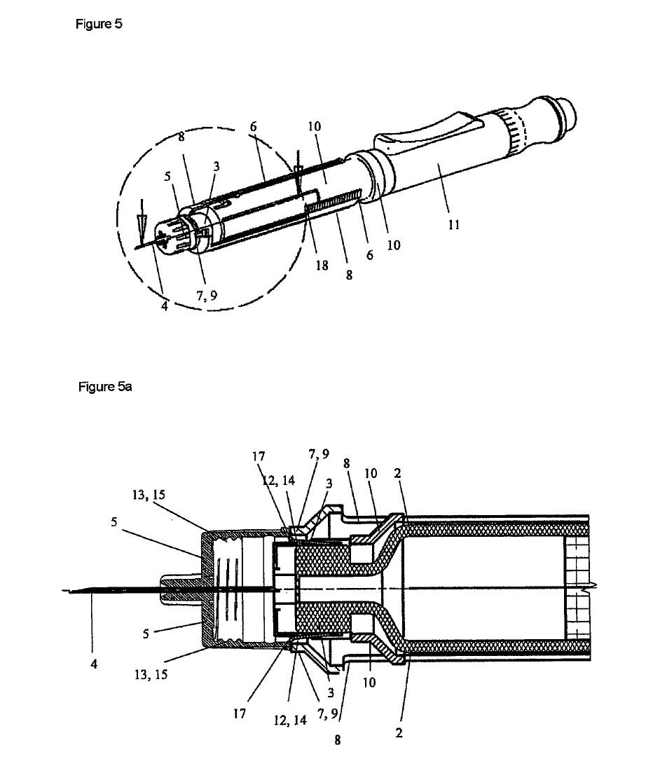

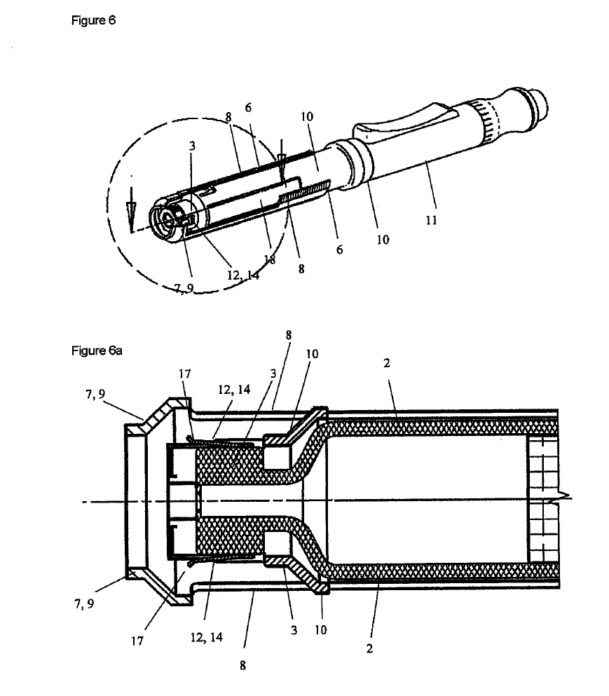

The main individual parts of the injection pen are shown in an exploded view in FIG. 3, while the functional relationship of the individual parts is shown in sectional views in FIGS. 4 and 6. In FIGS. 4, 5 and 6, the injection pen is shown in a perspective view at different stages during the release of the needle holder 5 from the needle seat 3. A quarter of the sleeve 8 and of the needle holder 5 has been cut away here in order to provide a plan view of the needle seat at these stages. The figures with the suffix “a” show a cross-sectional view of the injection pen in the area of the needle seat 3.

FIG. 4 shows the injection pen from FIG. 2 in a perspective view, with a partial sectional view of the sleeve 8 and of the needle holder 5. As has already been mentioned, the sleeve 8 is movable in a guide in the axial direction of the injection pen. FIGS. 6 and 6a show it in an end position in which the needle holder 5 sits securely on the needle seat 2. The secure fit is ensured by a projection 17, arranged on the spring element 14, engaging in a recess 15 of the needle holder 5.

In the position shown in FIGS. 4 and 4a, the injection pen is ready to inject a dose of medicament that has been set by means of the dosing mechanism 11. After the injection, the sleeve 8, the needle holder 5 and the spring elements 14 are still in the position shown in FIGS. 4 and 4a. However, after the dose set by the dosing mechanism 11 has been injected, it is necessary to dispose of the used needle 2. This is done by the needle holder 5 and needle 2, designed together as a disposable unit, being removed from the needle seat 3 and replaced by a new one.

In the described illustrative embodiment, the actuating element 6 arranged on the sleeve 8 is activated for this purpose. By means of the actuating element 6, the entire sleeve 8 is moved in the axial direction of the injection pen toward the needle holder 8. The annular release element 7 arranged on the sleeve 8 engages behind the needle holder 5 and pushes it from the needle seat. The axial movement of the sleeve 8 can take place via a linear guide arranged in the axial direction on the cartridge holder 10. However, it is also possible for an inner thread of the sleeve 8 to engage with an outer thread of the cartridge holder 10, thereby guiding the sleeve 8 and moving the latter in the axial direction of the injection pen. FIGS. 5 and 5a show a stage during the movement of the sleeve. FIG. 5a in particular shows how the projection 17 arranged on the spring element disengages from the recess 15 and the release of the needle holder from the needle seat is thus initiated.

The guide for the sleeve 8 is provided with abutments to prevent complete release of the sleeve 8 from the cartridge holder 10. The positions of the sleeve 6 at these abutments correspond to the positions shown in FIGS. 4 and 6 and in FIGS. 4a and 4a. In the position according to FIGS. 8 and 8a, the sleeve 8 has already moved as far as the abutment on which the release element 7 has pushed the needle holder 5 completely from the needle seat. The needle holder 8 is now safely released from the needle seat 3 and is disposed of in a disposal container that is made ready during the release procedure.

After this release, the sleeve 8 can be guided manually to the starting position. Alternatively, it is of course possible for an automated restoring mechanism, for example in the form of a restoring spring, to be integrated into the injection pen.

A new, unused needle holder 5 can now be arranged on the needle seat 3 in the usual way. It is of course also possible, and advantageous for hygiene reasons, to fit the protective cap 16 onto the injection pen and to arrange the new needle holder 10 on the needle seat 3 only just before a renewed injection.

### LIST OF REFERENCE SIGNS

1 housing part
2 cartridge
3 needle seat
4 needle
5 needle holder
6 actuating element
7 release element
8 sleeve
9 limit
10 cartridge holder
11 dosing mechanism
12 coupling means
13 mating coupling means
14 spring element
15 recess
16 protective cap
17 projection
18 viewing window
19 protective cap

1.-23. (canceled)

24. A device for injecting liquids, more particularly liquid medicaments or hormone preparations, for example insulin, said device comprising
a housing part
in or on which a cartridge containing the liquid can be arranged,
wherein the housing part or the cartridge has a needle seat
designed for releasable attachment of a needle holder carrying a needle, and
wherein the cartridge , upon release of the needle holder from the needle seat, is held fixed in position in or on the housing part,
characterized in that,
in order to release the needle holder, a release element is provided that engages on the needle holder and that can be activated by an actuating element.

25. The device as claimed in claim 24, characterized in that it is designed for manual release of the needle holder.

26. The device as claimed in claim 24, characterized in that it is designed for the needle holder to be released by motor, more particularly by electric motor.

27. The device as claimed in claim 24, characterized in that the housing part is substantially pen-shaped or axially symmetrical with respect to the longitudinal axis thereof.

28. The device as claimed in claim 24, characterized in that it is designed as an injection pen.

29. The device as claimed in claim 24, characterized in that the release element is arranged on the housing part so as to be axially movable.

30. The device as claimed in claim 24, characterized in that the release element and the actuating element are connected to each other rigidly or via gear mechanisms.

31. The device as claimed in claim 29, characterized in that the release element and/or the actuating element can be moved along a guide on the housing part.

32. The device as claimed in claim 31, characterized in that the guide is designed as at least one linear guide rail in the axial direction of the housing part, and elements for engagement in the at least one guide rail are arranged on the release element and/or the actuating element.

33. The device as claimed in claim 31, characterized in that the guide is formed by an outer thread that is arranged on the housing part and that engages with an inner thread of the release element and/or of the actuating element.

34. The device as claimed in claim 31, characterized in that a sleeve that can be moved axially on the housing part is provided as release element and/or actuating element .

35. The device as claimed in claim 24, characterized in that the release element for releasing the needle holder engages the latter with a form fit and/or frictional fit and transmits its axial movement to the needle holder.

36. The device as claimed in claim 24, characterized in that the release element has an annular limit which, in the position of use, engages behind the needle holder arranged on the needle seat.

37. The device as claimed in claim 24, characterized in that the release element and/or the actuating element can be fixed by at least one fixing element, so as to avoid unintentional release of the needle holder from the cartridge holder.

38. The device as claimed in claim 24, characterized in that the housing part is designed as cartridge holder.

39. The device as claimed in claim 24, characterized in that the housing part has a dosing mechanism.

40. The device as claimed in claim 38, characterized in that the cartridge holder and the dosing mechanism can be connected to each other in a releasable manner by means of a coupling.

41. The device as claimed in claim 24, characterized by the housing part and/or the cartridge holder and/or the sleeve having an opening, such that the filling level of a cartridge made of transparent material can be monitored.

42. The device as claimed in claim 24, characterized in that the housing part or the cartridge has coupling means, which correspond with mating coupling means on the needle holder.

43. The device as claimed in claim 42, characterized in that the coupling means are designed as spring elements , and the mating coupling means are designed as recesses which correspond thereto and into which the spring elements engage.

44. The device as claimed in claim 42, characterized in that the coupling means and the mating coupling means are designed as locking elements and mating locking elements, respectively, or as clip elements and mating clip elements, respectively.

45. The device as claimed in claim 42, characterized in that the coupling means and the mating coupling means are designed as outer and inner threads.

46. The device as claimed in claim 24, characterized in that a protective cap is provided for the housing part or the cartridge holder.
